(19) United States
(12) Patent Application Publication
(10) Pub. No.: US 2020/0261445 A1
(43) Pub. Date: Aug. 20, 2020

(54) USE OF SENOLYTIC AGENTS TO ELIMINATE PERSISTENT HIV RESERVOIRS

(71) Applicant: The George Washington University, Washington, DC (US)

(72) Inventors: R. Brad JONES, Washington, DC (US); John HUANG, Washington, DC (US); Yanqin REN, Washington, DC (US)

(21) Appl. No.: 16/651,470

(22) PCT Filed: Sep. 28, 2018
(86) PCT No.: PCT/US2018/053541

§ 371 Date: Mar. 27, 2020

Related U.S. Application Data
(60) Provisional application No. 62/565,406, filed on Sep. 29, 2017.

(51) Int. Cl. A61K 31/496; A61K 31/4725; A61K 35/17; A61K 31/365; A61P 31/18

(52) U.S. Cl. CPC A61K 31/496; A61K 31/4725; A61K 45/06; A61K 31/365; A61P 31/18; A61K 35/17

(57) ABSTRACT

The present disclosure provides methods of reducing or eliminating HIV reservoirs in a human infected with HIV, comprising administering to the human a therapeutically effective amount of a Bcl-2 family inhibitor and one or more additional agents. Additional agents include a therapeutically effective amount of a latency reversing agent, a therapeutically effective vaccine which enhances CTL responses, an immunotherapy which enhances CTL responses, a therapeutically effective vaccine which enhances CD8+ T-cell responses, an immunotherapy which enhances CD8+ T-cell responses, a CTL, or a CD8+ T-cell.

### STATEMENT OF GOVERNMENTAL INTEREST

This invention was made with government support under grant number UM1 AI126617 awarded by the National Institutes of Health. The government has certain rights in this invention.

### TECHNICAL FIELD

This disclosure relates to the eradication of HIV reservoirs in HIV patient.

### BACKGROUND OF THE INVENTION

Antiretroviral (ARV) drug regimens effectively suppress HIV replication, but are unable to cure infection. The persistence of virus leaves even well-treated individuals with a lifelong commitment to drug regimens, burdened by co-morbidities, such as cardiovascular disease and neurocognitive disorders, and exposed to the social stigma that comes with being HIV-positive (Deeks et al., Lancet 382:1525-1533 (2013); Rueda et al., Curr Opin HIV AIDS 9:325-331 (2014)).

The persistence of HIV in ARV-treated individuals is facilitated primarily by the establishment of latent infection in resting CD4⁺ T-cells. While in this latent state, HIV-infected resting CD4⁺ T-cells do not express virus, and thus neither die by viral cytopathic effects nor are eliminated by the immune system. HIV can be reactivated from latency by a variety of biological and pharmacological agents. However, reactivation alone has been shown to be insufficient to cause the death of infected cells. This has led to the emergence of “kick and kill” as the dominant paradigm in HIV cure research. Kick and kill strategies propose to combine a latency-reversing agent (LRA) with immune effectors, such as a CD8⁺ T-cells or NK cells, to selectively eliminate infected cells from the reservoir (Shan et al., Immunity 36:491-501 (2012); Deeks Nature 487:439-440 (2012); Archin and Margolis, Curr Opin Infect Dis. 27:29-35 (2014)).

The persistence of latently infected cells in HIV patients under combinatory antiretroviral therapy is a key obstacle to HIV eradication. There exists a need to purge these reservoirs in HIV patients.

### BRIEF SUMMARY OF THE INVENTION

In one aspect, the present disclosure provides a method of reducing or eliminating HIV reservoirs in a human infected with HIV, comprising administering to the human a therapeutically effective amount of a Bcl-2 family inhibitor and a therapeutically effective amount of a latency reversing agent.

In another aspect, the present disclosure provides a method of reducing or eliminating HIV reservoirs in a human infected with HIV, comprising administering to the human a therapeutically effective amount of a Bcl-2 family inhibitor and a therapeutically effective amount of CD8+ T-cells.

In another aspect, the present disclosure provides a method of reducing or eliminating HIV reservoirs in a human infected with HIV, comprising administering to the human a therapeutically effective amount of a Bcl-2 family inhibitor and a therapeutically effective amount of cytotoxic T-lymphocytes (CTLs).

In another aspect, the present disclosure provides a method of reducing or eliminating HIV reservoirs in a human infected with HIV, comprising administering to the human a therapeutically effective amount of a Bcl-2 family inhibitor, a therapeutically effective amount of a latency reversing agent, and a therapeutically effective amount of a CD8+ T-cells.

In another aspect, the present disclosure provides a method of reducing or eliminating HIV reservoirs in a human infected with HIV, comprising administering to the human a therapeutically effective amount of a Bcl-2 family inhibitor, a therapeutically effective amount of a latency reversing agent, and a therapeutically effective amount of a cytotoxic T-lymphocyte. In another aspect, a latency reversing agent can be omitted.

In another aspect, the present disclosure provides a method of reducing or eliminating HIV reservoirs in a human infected with HIV, comprising administering to the human a therapeutically effective amount of a Bcl-2 family inhibitor and a therapeutically effective vaccine which enhances CD8+ T-cell responses.

In another aspect, the present disclosure provides a method of reducing or eliminating HIV reservoirs in a human infected with HIV, comprising administering to the human a therapeutically effective amount of a Bcl-2 family inhibitor and a therapeutically effective vaccine which enhances CTL responses.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### BRIEF DESCRIPTION OF THE DRAWINGS

FIG. 1 is a bar graph showing droplet digital PCR (ddPCR) results from an HIV eradication (HIVE) assay for senolytic and LRA combinations (ABT-199 at 1 μM, A-463 at 100 nM, and A-852 at 100 nM). “A-463” refers to A-1155463 and “A-852” refers to A-1331852. “Bryo” refers to bryostatin.

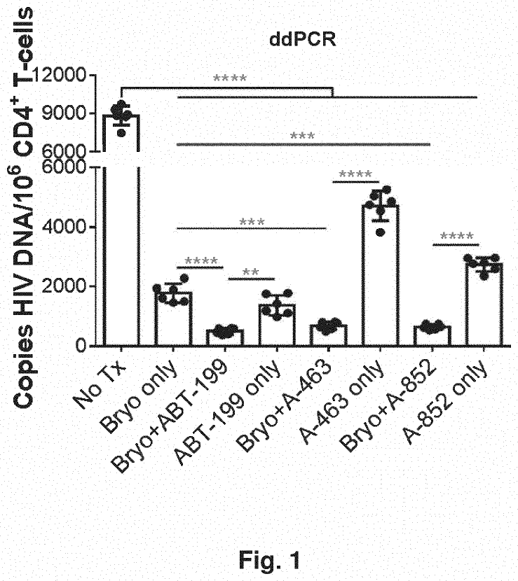

FIG. 2 is a bar graph showing quantitative viral outgrowth (QVOA) analysis of CD4+ T-cells post-HIVE assay for senolytic and LRA combinations (ABT-199 at 1 μM, A-463 at 100 nM, and A-852 at 100 nM). “A-463” refers to A-1155463 and “A-852” refers to A-1331852. “Bryo” refers to bryostatin.

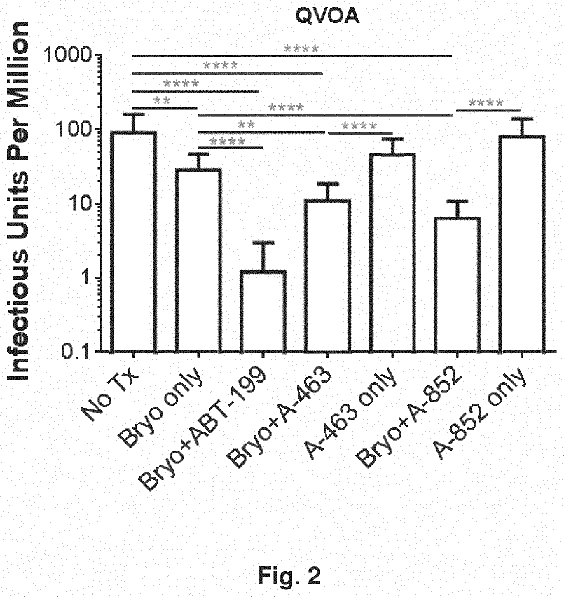

FIG. 3 is a bar graph showing ddPCR results from an HIVE assay for senolytic and LRA combinations. “A-463” refers to A-1155463 and “A-852” refers to A-1331852. “Bryo” refers to bryostatin. “Vori” refers to vorinostat. “Romi” stands for romidepsin.

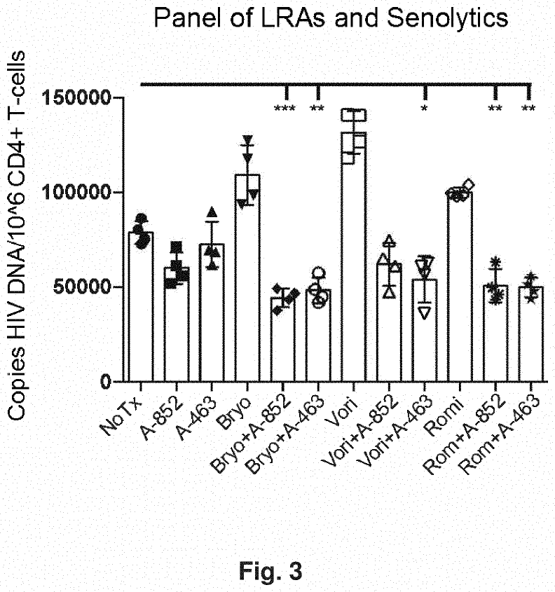

FIG. 4 is a bar graph showing ddPCR results from an HIVE assay with or without NK cells for a ABT-199 (senolytic) and bryostatin (LRA) combination.

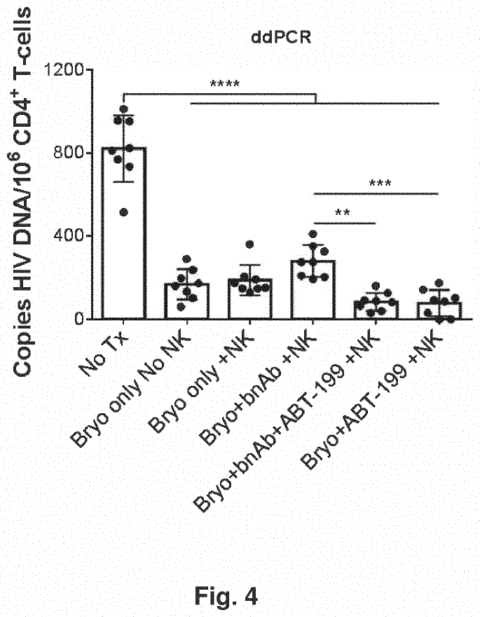

FIG. 5 is a bar graph showing QVOA analysis of CD4+ T-cells post-HIVE assay with or without NK cells for a ABT-199 and bryostatin combination.

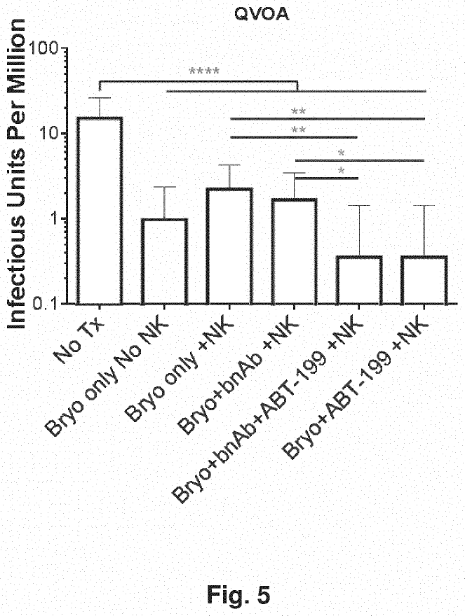

FIG. 6 is a bar graph showing ddPCR results from HIVE assay for a ABT-199, bryostatin, and HIV-specific cytotoxic T lymphocyte (CTL) combination.

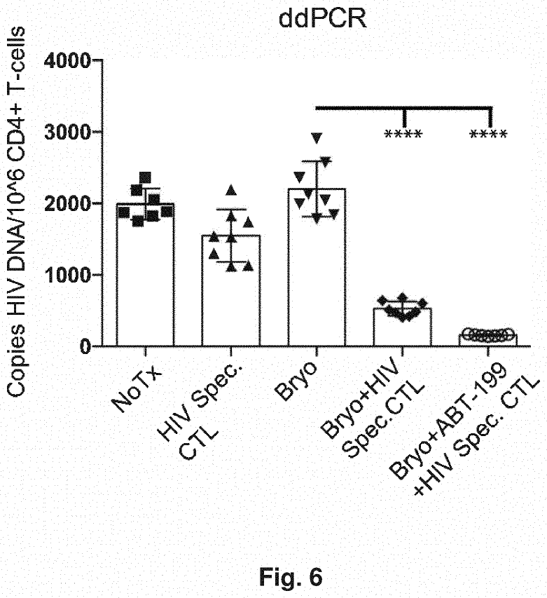

FIG. 7 is a bar graph showing QVOA analysis post-HIVE assay for a ABT-199, bryostatin, and HIV-specific cytotoxic T lymphocyte (CTL) combination.

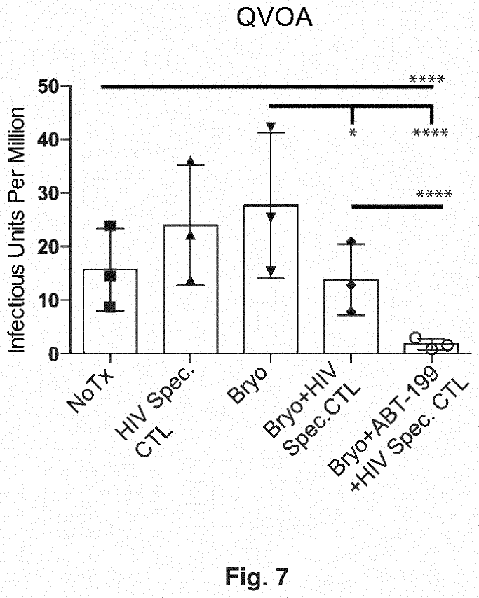

FIG. 8 is a bar graph showing QVOA analysis of CD4+ T-cells post-HIVE assay for a CD3/CD28, ABT-199, and HIV-specific CTL combination.

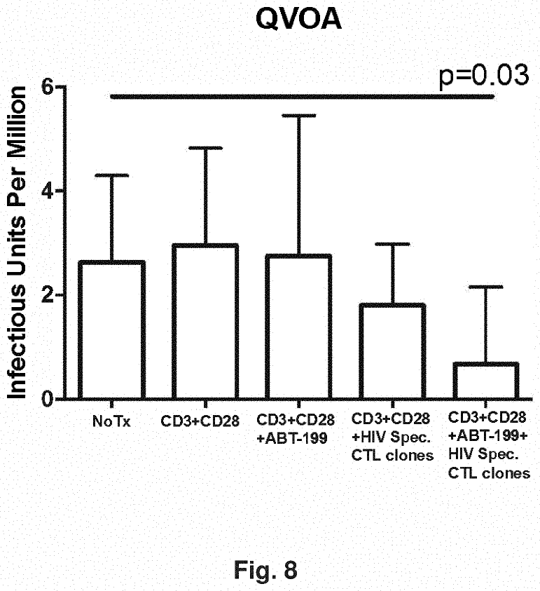

FIG. 9A is a line chart showing the average copies of HIV DNA per million CD4⁺ T-cells following treatment with 1 μM ABT-199 (ABT), bryostatin, or a combination of bryostatin and 1 μM ABT-199.

FIG. 9B is a line chart showing the infectious units per million cells (IUPM) following treatment with 1 μM ABT-199 (ABT), bryostatin, or a combination of bryostatin and 1 μM ABT-199.

FIG. 9C is a line chart showing the average copies of HIV DNA per million CD4⁺ T-cells following treatment with 100 nM ABT-199 (ABT), bryostatin, or a combination of bryostatin and 100 nM ABT-199.

FIG. 9D is a line chart showing the infectious units per million cells (IUPM) following treatment with 100 nM ABT-199 (ABT), bryostatin, or a combination of bryostatin and 100 nM ABT-199.

FIG. 9E is a line chart showing the average copies of HIV DNA per million CD4⁺ T-cells following treatment with 100 nM A-463, bryostatin, or a combination of bryostatin and 100 nM A-463.

FIG. 9F is a line chart showing the infectious units per million cells (IUPM) following treatment with 100 nM A-463, bryostatin, or a combination of bryostatin and 100 nM A-463.

FIG. 10 is a bar graph showing QVOA analysis post-HIVE assay for a ABT-199, bryostatin, and HIV-specific cytotoxic T lymphocyte (CTL), and combinations thereof. Assay results show median ±95%, and p values were calculated using a chi-squared test. (*** p<0.001, **** p<0.0001; relative to the bryostatin+HIV-specific CTL+ABT-199 condition).

FIG. 11A is a line chart showing the infectious units per million cells (IUPM) for an individual HIVE assay before and after treatment with CD3+CD28 antibody.

FIG. 11B is a line chart showing the infectious units per million cells (IUPM) for an individual HIVE assay before and after treatment with CD3+CD28 antibody and HIV-specific T cell lines.

FIG. 11C is a line chart showing the infectious units per million cells (IUPM) for an individual HIVE assay before and after treatment with CD3+CD28 antibody and ABT-199.

FIG. 11D is a line chart showing the infectious units per million cells (IUPM) for an individual HIVE assay before and after treatment with CD3+CD28 antibody, ABT-199, and HIV-specific T cell lines.

FIG. 12 is a bar graph showing the average fold decrease in IUPM ±SD between the indicated treatment condition and the NoTx condition. The dotted line indicates a 1-fold decrease (no decrease).

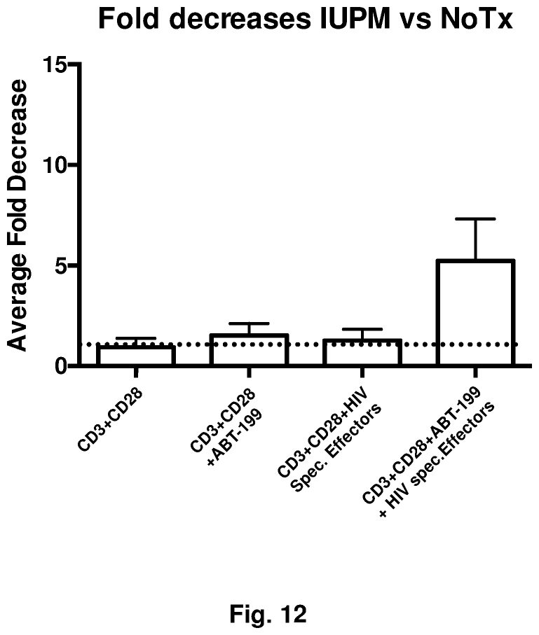

### DETAILED DESCRIPTION OF THE INVENTION

In one embodiment, the present disclosure provides a method of reducing or eliminating HIV reservoirs in a human infected with HIV, comprising administering to the human a therapeutically effective amount of a Bcl-2 family inhibitor and a therapeutically effective amount of a latency reversing agent.

In another embodiment, the present disclosure provides a method of reducing or eliminating HIV reservoirs in a human infected with HIV, comprising administering to the human a therapeutically effective amount of a Bcl-2 family inhibitor and a therapeutically effective amount of a latency reversing agent, wherein the Bcl-2 family inhibitor is a selective Bcl-2/Bcl-xL inhibitor.

In another embodiment, the present disclosure provides a method of reducing or eliminating HIV reservoirs in a human infected with HIV, comprising administering to the human a therapeutically effective amount of a Bcl-2 family inhibitor and a therapeutically effective amount of a latency reversing agent, wherein the Bcl-2 family inhibitor is a Bcl-2 inhibitor. In another embodiment, the Bcl-2 inhibitor is a selective Bcl-2 inhibitor. In another embodiment, the selective Bcl-2 inhibitor is ABT-199.

In another embodiment, the present disclosure provides a method of reducing or eliminating HIV reservoirs in a human infected with HIV, comprising administering to the human a therapeutically effective amount of a Bcl-2 family inhibitor and a therapeutically effective amount of a latency reversing agent, wherein the Bcl-2 family inhibitor is a Bcl-xL inhibitor. In another embodiment, the Bcl-xL inhibitor is a selective Bcl-xL inhibitor. In another embodiment, the selective Bcl-xL inhibitor is A-1155463 or A-1331852.

In another embodiment, the present disclosure provides a method of reducing or eliminating HIV reservoirs in a human infected with HIV, comprising administering to the human a therapeutically effective amount of a Bcl-2 family inhibitor and a therapeutically effective amount of a latency reversing agent, wherein the Bcl-2 family inhibitor is selected from the group consisting of ABT-263, ABT-737, sabutoclax, AT-101, TW-37, gambogic acid, BH31-1, ABT-199, obatoclax, HA14-1, A-1155463, A-1331852, WEHI-539, A-1210477, and UMI-77. In another embodiment, the present disclosure provides a method of reducing or eliminating HIV reservoirs in a human infected with HIV, comprising administering to the human a therapeutically effective amount of a Bcl-2 family inhibitor and a therapeutically effective amount of a latency reversing agent, wherein the latency reversing agent is selected from the group consisting of a protein kinase C activator, a histone deacytelase inhibitor, a Toll-like receptor 2 agonist, a gamma chain cytokine, and a P-TEFb inhibitor.

In another embodiment, the present disclosure provides a method of reducing or eliminating HIV reservoirs in a human infected with HIV, comprising administering to the human a therapeutically effective amount of a Bcl-2 family inhibitor and a therapeutically effective amount of a latency reversing agent, wherein the latency reversing agent is a protein kinase C activator.

In another embodiment, the present disclosure provides a method of reducing or eliminating HIV reservoirs in a human infected with HIV, comprising administering to the human a therapeutically effective amount of a Bcl-2 family inhibitor and a therapeutically effective amount of a latency reversing agent, wherein the latency reversing agent is a histone deacytelase inhibitor.

In another embodiment, the present disclosure provides a method of reducing or eliminating HIV reservoirs in a human infected with HIV, comprising administering to the human a therapeutically effective amount of a Bcl-2 family inhibitor and a therapeutically effective amount of a latency reversing agent, wherein the latency reversing agent is a Toll-like receptor 2 agonist.

In another embodiment, the present disclosure provides a method of reducing or eliminating HIV reservoirs in a human infected with HIV, comprising administering to the human a therapeutically effective amount of a Bcl-2 family inhibitor and a therapeutically effective amount of a latency reversing agent, wherein the latency reversing agent is a gamma chain cytokine.

In another embodiment, the present disclosure provides a method of reducing or eliminating HIV reservoirs in a human infected with HIV, comprising administering to the human a therapeutically effective amount of a Bcl-2 family inhibitor and a therapeutically effective amount of a latency reversing agent, wherein the latency reversing agent is a P-TEFb inhibitor. In another embodiment, the latency reversing agent is selected from the group consisting of bryostatin 1, prostratin, ingenol B, vorinostat, romidepsin, panobinostat, Pam3CSK4, ALT-803, IL-2, IL-7, IL-15, heterodimeric IL-15, IL-15N72D-IL-15 receptor alpha Su/Fc fusion protein, JQ1, and I-BET151. In another embodiment, the latency reversing agent is bryostatin 1.

In another embodiment, the present disclosure provides a method of reducing or eliminating HIV reservoirs in a human infected with HIV, comprising administering to the human a therapeutically effective amount of a Bcl-2 inhibitor, a therapeutically effective amount of a latency reversing agent, and a therapeutically effective amount of CD8+ T-cells.

In another embodiment, the present disclosure provides a method of reducing or eliminating HIV reservoirs in a human infected with HIV, comprising administering to the human a therapeutically effective amount of a Bcl-2 inhibitor, a therapeutically effective amount of a latency reversing agent, and a therapeutically effective amount of a cytotoxic T-lymphocyte.

In another embodiment, the present disclosure provides a method of reducing or eliminating HIV reservoirs in a human infected with HIV, comprising administering to the human a therapeutically effective amount of a Bcl-2 inhibitor and a therapeutically effective amount of a cytotoxic T-lymphocyte.

In another embodiment, the present disclosure provides a method of reducing or eliminating HIV reservoirs in a human infected with HIV, comprising administering to the human a therapeutically effective amount of a Bcl-2 inhibitor and a therapeutically effective vaccine which enhances CD8+ T-cell responses.

In another embodiment, the present disclosure provides a method of reducing or eliminating HIV reservoirs in a human infected with HIV, comprising administering to the human a therapeutically effective amount of a Bcl-2 inhibitor and a therapeutically effective vaccine which enhances CTL responses.

In another embodiment, the present disclosure provides a method of reducing or eliminating HIV reservoirs in a human infected with HIV, comprising administering to the human a therapeutically effective amount of a Bcl-2 inhibitor, a therapeutically effective amount of a latency reversing agent, and a therapeutically effective vaccine which enhances CD8+ T-cell responses.

In another embodiment, the present disclosure provides a method of reducing or eliminating HIV reservoirs in a human infected with HIV, comprising administering to the human a therapeutically effective amount of a Bcl-2 inhibitor, a therapeutically effective amount of a latency reversing agent, and a therapeutically effective vaccine which enhances CTL responses.

In another embodiment, the present disclosure provides a method of reducing or eliminating HIV reservoirs in a human infected with HIV, comprising administering to the human a therapeutically effective amount of a Bcl-2 inhibitor and a therapeutically effective immunotherapy which enhances CD8+ T-cell responses.

In another embodiment, the present disclosure provides a method of reducing or eliminating HIV reservoirs in a human infected with HIV, comprising administering to the human a therapeutically effective amount of a Bcl-2 inhibitor and a therapeutically effective immunotherapy which enhances CTL responses.

In another embodiment, the present disclosure provides a method of reducing or eliminating HIV reservoirs in a human infected with HIV, comprising administering to the human a therapeutically effective amount of a Bcl-2 inhibitor, a therapeutically effective amount of a latency reversing agent, and a therapeutically effective immunotherapy which enhances CD8+ T-cell responses.

In another embodiment, the present disclosure provides a method of reducing or eliminating HIV reservoirs in a human infected with HIV, comprising administering to the human a therapeutically effective amount of a Bcl-2 inhibitor, a therapeutically effective amount of a latency reversing agent, and a therapeutically effective immunotherapy which enhances CTL responses.

The disclosure also provides the following particular embodiments.

### Embodiment I

A method of reducing or eliminating HIV reservoirs in a human infected with HIV, the method comprising administering to the human a therapeutically effective amount of a Bcl-2 family inhibitor and a therapeutically effective amount of a latency reversing agent.

### Embodiment II

The method of Embodiment I, wherein the Bcl-2 family inhibitor is a Bcl-2 inhibitor.

### Embodiment III

The method of Embodiment II, wherein the Bcl-2 inhibitor is a selective Bcl-2 inhibitor.

### Embodiment IV

The method of Embodiment I, wherein the Bcl-2 family inhibitor is a Bcl-xL inhibitor.

### Embodiment V

The method of Embodiment IV, wherein the Bcl-xL inhibitor is a selective Bcl-xL inhibitor.

### Embodiment VI

The method of Embodiment I, wherein the Bcl-2 family inhibitor is a selective Bcl-2/Bcl-xL inhibitor.

### Embodiment VII

The method of Embodiment II, wherein the Bcl-2 family inhibitor is selected from the group consisting of ABT-263, ABT-737, sabutoclax, AT-101, TW-37, gambogic acid, BH31-1, ABT-199, obatoclax, HA14-1, A-1155463, A-1331852, WEHI-539, A-1210477, and UMI-77.

### Embodiment VIII

The method of Embodiment VII, wherein the Bcl-2 family inhibitor is ABT-199.

### Embodiment IX

The method of Embodiment VII, wherein the Bcl-2 family inhibitor is A-1155463 or A-1331852.

### Embodiment X

The method of any one of Embodiments I-IX, wherein the latency reversing agent is selected from the group consisting of protein kinase C activator, histone deacytelase inhibitor, Toll-like receptor 2 agonist, gamma chain cytokine, and P-TEFb inhibitor.

### Embodiment XI

The method of any one of claims I-IX, wherein the latency reversing agent is selected from the group consisting of bryostatin 1, prostratin, ingenol B, vorinostat, romidepsin, panobinostat, Pam3CSK4, ALT-803, IL-2, IL-7, IL-15, heterodimeric IL-15, IL-15N72D-IL-15 receptor alpha Su/Fc fusion protein, JQ1, and I-BET151.

### Embodiment XII

The method of any one of Embodiment I-X, wherein the latency reversing agent is a protein kinase C activator.

### Embodiment XIII

The method of Embodiment XII, wherein the protein kinase C activator is bryostatin 1.

### Embodiment XIV

The method of any one of Embodiment I-XIII further comprising administering a therapeutically effective amount of cytotoxic T-lymphocytes to the human.

### Definitions

The term “HIV reservoir” as used herein refers to cells infected with replication-competent HIV-1 that persists on long timescales (from years to indefinitely) in patients, despite immune pressure and optimal antiretroviral therapy. (Eisele et al, Immunity. 37(3):377-88 (2012)).

The term “Bcl-2 family protein” as used herein refers to any one or more of the following proteins: Bax, Bak, Bid, Bcl-2, Bcl-xL, Mcl-1, Bcl-w, Bfl-1/A1, Bim, Puma, Bad, Bik/Blk, Noxa, Bmf, Hrk/DP5, and Beclin-1. See Cold Spring Harb Perspect Biol 2013; 5:a008714. Anti-apoptotic members of this family include Bcl-2 and Bcl-xL.

The term “Bcl-2 family inhibitor” as used herein refers to a compound that inhibits any one or more of the following proteins: Bax, Bak, Bid, Bcl-2, Bcl-xL, Mcl-1, Bcl-w, Bfl-1/A1, Bim, Puma, Bad, Bik/Blk, Noxa, Bmf, Hrk/DP5, and Beclin-1.

Bcl-2 family inhibitors useful in the present disclosure include, but are not limited to, ABT-263, ABT-737, sabutoclax, AT-101, TW-37, gambogic acid, BH31-1, ABT-199 (Venetoclax), obatoclax, HA14-1, A-1155463, A-1331852, WEHI-539, A-1210477, or UMI-77. See Table 1.

TABLE 1

| | | | | Known |
|---|---|---|---|---|
| | | | BH3 | Bcl-xL |
| Category | Name | Selective/pan | mimetic? | inhibition |
| Pan Bcl-2 family | ABT-263 | Pan | | Yes |
| inhibitors | ABT-737 | Pan | Yes | Yes |
| (inhibit more than | Sabutoclax | Pan | | Yes |
| one of Bcl-xL, | AT101 | Pan | | Yes |
| Bcl-2, Bcl-W, | TW-37 | Pan | | Yes |
| Bcl-B, Mcl-1 | Gambogic | Pan | | Yes |
| | Acid | | | |
| and Bfl-1) | BH31-1 | Pan | Yes | Yes |
| Bcl-2 inhibitors | ABT-199 | selective | | Weak |
| | Obatoclax | Inhibition of | | |
| | | other Bcl-2 | | |
| | | family | | |
| | | members | | |
| | | possible | | |
| | HA14-1 | Inhibition of | | |
| | | other Bcl-2 | | |
| | | family members | | |
| | | possible | | |
| | A-1155463 | Selective | Yes | |
| Bcl-xL inhibitors | A-1331852 | Selective | Yes | |
| | WEHI-539 | Selective | | |
| MCL-1 | A-1210477 | Selective | | |
| | UMI-77 | Selective | | |

Bcl-2 family inhibitors are also referred to as “senolytics” because they, inter alia, cause the death of senescent cells.

The term “Bcl-xL inhibitor” as used herein refers to a compound that inhibits the anti-apoptotic Bcl-xL protein.

The term “Bcl-2 inhibitor” as used herein refers to a compound that inhibits the anti-apoptotic Bcl-2 protein.

The term “selective Bcl-2/Bcl-xL inhibitor” as used herein refers to a compound that preferentially inhibits Bcl-2 and/or Bcl-xL protein over the other Bcl-2 family proteins.

The term “selective Bcl-2 inhibitor” as used herein refers to a compound that preferentially inhibits Bcl-2 protein over other Bcl-2 family proteins. A-199 is a representative selective Bcl-2 inhibitor.

The term “selective Bcl-xL inhibitor” as used herein refers to a compound that preferentially inhibits Bcl-xL protein over other Bcl-2 family proteins. A-1155463 and A-1331852 are representative selective Bcl-X_{L} inhibitors. (ACS Med. Chem, Lett, 2014, 5 (1.0):1088-1093; Sci Transl Med., 2015; 7(279):279ra40) A-1155463 is labeled as A-463 and A-1331852 is labeled as A-852 in the figures.

The term “latency reversing agent” or “LRA” as used herein refers to a compound that reactivates latent HIV virus. Certain classes of latency reversing agents are useful in the present disclosure. These classes include, but are not limited to, protein kinase C activators, histone deacytelase inhibitors, Toll-like receptor 2 agonists, gamma chain cytokines, or P-TEFb inhibitors. In another embodiment, the latency reversing agent is selected from the group consisting of a HDAC inhibitor, a phorbol ester, IL-2, and a bromodomain inhibitor. Specific LRAs useful in the present disclosure include, but are not limited to, bryostatin 1, prostratin, ingenol B, vorinostat, romidepsin, panobinostat, Pam3CSK4, ALT-803, IL-2, IL-7, IL-15, heterodimeric IL-15, IL-15N72D-IL-15 receptor alpha Su/Fc fusion protein, JQ1, or I-BET151. See Table 2.

TABLE 2

| Name | Mode of Reactivation |
|---|---|
| Bryostatin | Protein Kinase C (PKC) activator |
| Prostratin | PKC activator |
| Ingenol B | PKC Activator |
| Vorinostat | Histone deacytelase inhibitor |
| Romidepsin | Histone deacytelase inhibitor |
| Panobinostat | Histone deactyelase inhibitor |
| Pam₃CSK₄ | Toll-like receptor 2 agonist - PKC activator |
| ALT-803 (IL-15 | Gamma chain cytokines - PKC activator |
| Superagonist) | |
| (N72D mutant) | |
| IL-2 | Gamma chain cytokines - cell activators |
| IL-7 | Gamma chain cytokines - cell activators |
| IL-15 | Gamma chain cytokines - PKC activator |
| Heterodimeric IL-15 | Gamma chain cytokines - PKC activator |
| IL-15N72D-IL-15 | Gamma chain cytokines - PKC activator |
| Receptor alpha | |
| Su/Fc fusion | |
| protein | |
| JQ1 | P-TEFb inhibitor |
| I-BET151 | P-TEFb inhibitor |

The term “cytotoxic T-lymphocyte” or “CTL” as used herein refers to an immune cell, e.g., CD8+ T-cells, that kills cells infected with a virus.

The term “CD8+ T-cells” as used herein refers to a CD8-expressing lymphocyte that exerts functions other than or in addition to killing that can enhance clearance of cells infected with a virus. A specific example is the release of IFN-gamma which induces upregulation of MHC-I on infected cells making them better targets for immune-mediated clearance.

The term “vaccine which enhances CTL responses” as used herein describes any immunogenic composition that primes novel HIV-specific CTL responses, or increases the magnitude or functionality of existing responses, following in vivo administration. A number of different vaccine platforms have been shown to achieve these outcomes, including: i) vectors derived of: virus-like particles, poxviruses, adenoviruses, or cytomegalovirus which deliver HIV antigens (reviewed in PMCID: PMC5874651) or ii) dendritic cell vaccines which deliver HIV antigens (reviewed in PMCID: PMC5187785).

The term “immunotherapy which enhances CTL responses” as used herein comprises the treatment with any biologic agent which acts to enhance the function or magnitude of CTL responses, including: i) checkpoint inhibitors such as anti-PD-1, anti-PD-L1, anti-CTLA-4, and anti-Tim-3 (reviewed in PMID: 28990586) (ii) cytokines such as IL-15, IL-2, IL-12, IL-21, or derivaties (reviewed in: PMID 27325459, 22763176, and 22169717.

The term “therapeutically effective amount” as used herein refers to the amount of Bcl-2 inhibitor, LRA, and/or CTL sufficient deplete the HIV reservoir in a patient infected with HIV. For example, in one embodiment, a therapeutically effective amount refers to the amount Bcl-2 family inhibitor, e.g., a Bcl-2 inhibitor or a Bcl-xL inhibitor, and LRA that decreases the HIV reservoir at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%.

The terms “a” and “an” refer to one or more than one.

The term “about,” as used herein, includes the recited number ±10%. Thus, “about 10” means 9 to 11.

### EXAMPLES

### Example 1

Target Cell Preparation:

For each condition tested, up to 200×10⁶ PBMC were thawed from leukapheresis material. An aliquot of PBMCs was stained with antibodies to CD3, CD4, CD8, HLA-DR, and CD69 for flow cytometry analysis (pre-selection sample). Resting CD4⁺ T-cells were enriched from these PBMC by negative selection (Easysep CD4⁺ T-cell enrichment kit, Stemcell Technologies) following the manufacturer's instructions. Cells were washed and re-suspended in 1 ml of 2% FBS PBS and an aliquot was stained as above (post-selection sample). To proceed, >98% pure resting CD4⁺ T-cells in the negative fraction we required.

Preparation of Immune Effectors:

NK cells: ex vivo NK cells were isolated by negative selection from PBMCs autologous to the CD4⁺ targets (Easysep NK cell enrichment kit, Stemcell Technologies), then added to culture at 1:10 effector:target ratios.

Clone Preparation:

For experiments using CTL clones as effectors, clones were used at −3 weeks after their most recent re-stimulation. Specificity, functionality, and responsiveness of CTLs were confirmed by CD107a degranulation assays, as well as by HIV elimination assays the day before assay setup. Clones were washed extensively prior to addition to co-culture.

Co-Culture:

NK cells and CTL clones were added at 1:10 effector:target ratios, with or without the indicated latency reversing agents. Co-cultures were performed at 2×10⁶ cells/ml in XVIVO-15 serum free medium (Lonza) that had been supplemented with penicillin-streptomycin, L-glutamine, 0.1 nM IL-7 (to support survival), 1 μM tenofovir, 1 μM nevirapine, 1 μM emtricitabine, 10 μM T20, 10 U/ml DNAse I (ProSpec) (XVIVO-10+7+ARV). LRA concentrations are given below, and added using a pulse-wash procedure: targets were co-cultured with LRAs for 2 hours, and then transferred to XVIVO-10+7+ARV after washing 3 times to prevent LRA carryover. NK cells or CTL clones were then added to the co-culture.

Harvest:

Co-cultures were harvested at 4 days post-initiation of co-culture. Cells from each condition were pelleted at 500×g, then re-suspended and isolated by CD4+ T-cell negative selection as above. A second elution was performed and pooled with the first; the pooled mixture was then re-extracted in a magnetic field to remove residual labeled cells. Cells were then pelleted and re-suspended in RPMI supplemented with 10% FBS, penicillin/streptomycin, and 50 U/mL IL-2 (R10-50). Aliquots of pre- and post-CD4 enrichment were stained with the antibodies (CD3, CD4, CD8, CD45RA, CD27, CCR7) to check purity and memory phenotype, and CD3, CD4, CD8, CD69, HLA-DR, and amine aqua viability dye with counting beads to check activation phenotypes and obtain an accurate cell count. CD4⁺ T-cells were then fixed in 2% paraformaldehyde, and analyzed by flow cytometry on an LSR-II instrument and with FlowJo software.

Quantifying Remaining Reservoir for HIV:

DNA quantification: 2×10⁶ cells/condition were centrifuged and DNA, for ddPCR, was extracted from cell pellets using the Gentra Puregene kit (Qiagen), following the manufacturer's instructions. This DNA was then analyzed by digital droplet polymerase chain reaction (see below). Infectious virus quantification: The remaining cells, to be used for viral outgrowth assays, were incubated overnight in R10-50+4 nM IL-15SA (ALT-803) at a concentration of 2×10⁶ cells/ml. These cells are then plated out in quantitative viral outgrowth assays (see below).

Digital Droplet Polymerase Chain Reaction

Digital droplet PCR was performed as previously described (Strain et al. PLoS One 8, e55943 (2013)). Genomic DNA was extracted using the Gentra Puregene kit (Gentra) following the manufacturer's instructions. For each PCR reaction, 5 units of restriction enzyme BsaJI (NEB) was directly mixed with 300 ng of DNA, ddPCR Supermix for Probes (Bio-Rad), and final concentrations of 900 nM primers and 250 nM probe. Primers/Probes were: RPP30-fprimer GATTTGGACCTGCGAGCG (SEQ ID No. 1), rprimer GCGGCTGTCTCCACAAGT (SEQ ID No. 2), probe VIC-CTGAACTGAAGGCTCT (SEQ ID No. 3); Covalently attached to N-terminus of SEQ ID No.4, MGBNFQ (SEQ ID No. 4); covalently attached to 3′-end of SEQ ID No. 3; HIV-gag-fprimer TACTGACGCTCTCGCACC (SEQ ID No. 5), rprimer TCTCGACGCAGGACTCG (SEQ ID No. 6), probe FAM-CTCTCTCCTTCTAGCCTC (SEQ ID No. 7) Covalently attached to N-terminus of SEQ ID No. 8, MGBNFQ (SEQ ID No. 8); covalently attached to 3′ end of SEQ ID No. 7. Droplets were prepared using the QX100 Droplet Generator (Bio-Rad) following the manufacturer's instructions. Sealed plates were cycled using the following program: 95° C. for 10 min; 40 cycles of 94° C. for 30 s, 60° C. for 1 min; and 98° C. for 10 min, with 2° C./sec ramping speed to ensure even droplet heating. Reactions were analyzed using the QX100 Droplet Reader, and template molecules per μL of starting material were estimated using the Quantalife ddPCR software. We aimed to run 8 replicates of ddPCR per sample—depending on the amount of DNA availability. Pre-determined exclusion criteria to outliers that deviated from mean values by >2× the standard deviation were applied.

Viral Outgrowth Assays

Outgrowth assays were performed using a previously described protocol, with slight modifications. (Laird et al. PLoS pathogens 9, e1003398 (2013)). Following overnight culture, as described above, cells were washed 3× and plated at 3-4 dilutions, and 8-12 replicates per dilution, depending on estimated viral burden. The exact numbers of replicates and the cell numbers were based on numbers of cells recovered at the end of the co-culture period, and in some cases were modified to reflect the study participants' infectious virus burden (IUPM) (ex. higher cell numbers for individuals with lower IUPMs). All cells were stimulated with 2×10⁶ irradiated autologous feeder cells (HIV-negative donor), and 2 μg/ml of PHA (Sigma) per well. Following 24 hours of co-culture, 2×10⁶ MOLT-4 CCRS cells were added to each well along with a ˜50% media change. Cultures were then incubated for 14 days, with partial changes of yellowing medium (every 3-4 days). On day 14, we quantified p24 in supernatant by ELISA (Perkin Elmer), following the manufacturer's instructions. For each treatment condition, values for cells/well, number of positive wells, and total wells plated were entered into a limiting dilution analyzer (either http://bioinf.wehi.edu.au/software/elda/ or http://silicianolab.johnshopkins edu) to calculate the probable ranges of IUPM using 95% confidence intervals.

Latency Reversing Agents

LRAs were used at the following concentrations: romidepsin at 40 nM, suberoylanilide hydroxamic acid at 335 nM (SAHA or vorinostat) (Sigma-Aldrich), bryostatin at 10 nM (Sigma-Aldrich), and CD3/CD28 antibodies at 1 μg/mL. Romidepsin, SAHA, and bryostatin were dissolved in hybrimax DMSO (Sigma-Aldrich). CD3/CD28 antibodies were stored in PBS. Stocks of the above reagents (minus CD3/CD28 antibodies) were flash-frozen in single-use aliquots in EtOH dry-ice baths, and stored at −20° C. The IL-15SA (ALT-803) was stored at 4° C. in PBS, and used at 144 ng/mL.

### Example 2

Three HIV latency reservoir models: 1) natural reservoir (resting CD4⁺ T cells) directly from HIV⁺ long-term ART donors (triangle symbols); 2) lab-made HIV latency reservoir cells only (open circle symbols) or 3) spiked into natural reservoirs (black dot symbols) were tested with the Bcl-2 inhibitor ABT-199 and the Bcl-xL inhibitor A-1155463 (A-463). See Bosque and Planelles, Blood 113:58-65 (2009); Bosque and Planelles, Methods 53: 54-61 (2011); and Novis et al., Retrovirology 10:119 (2013).

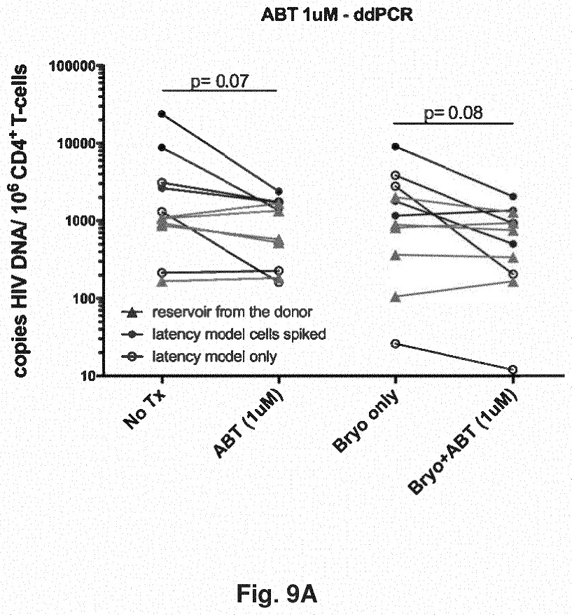

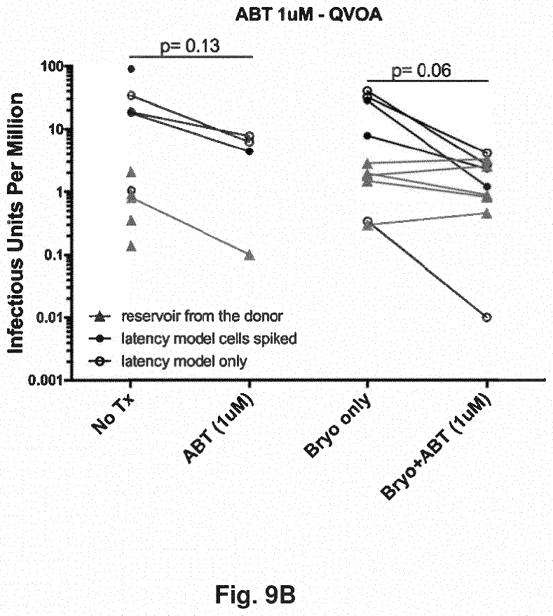

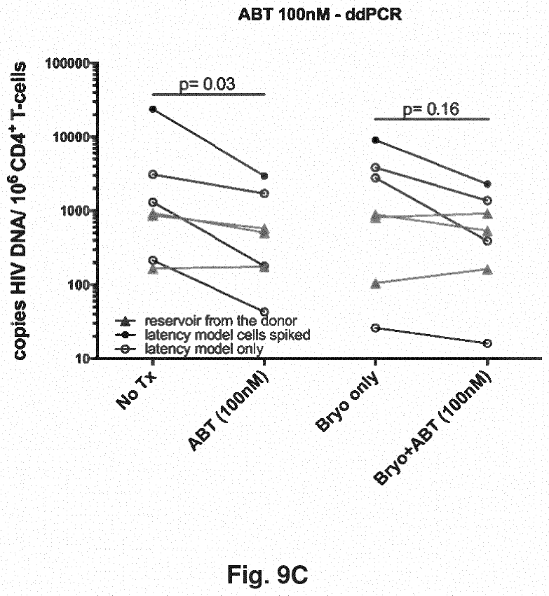

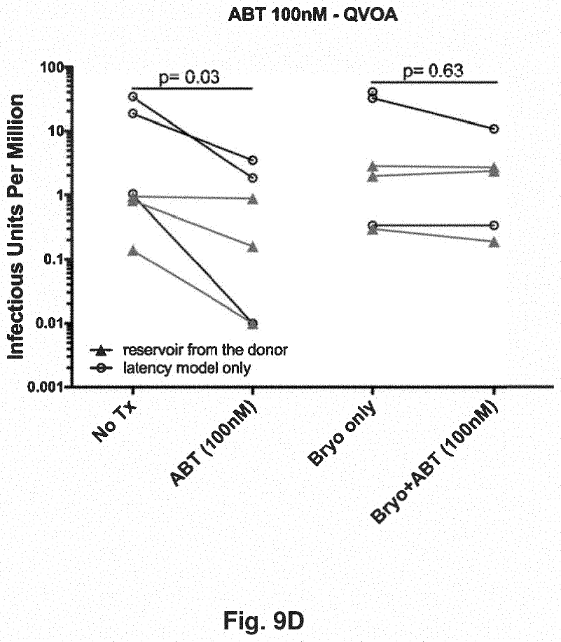

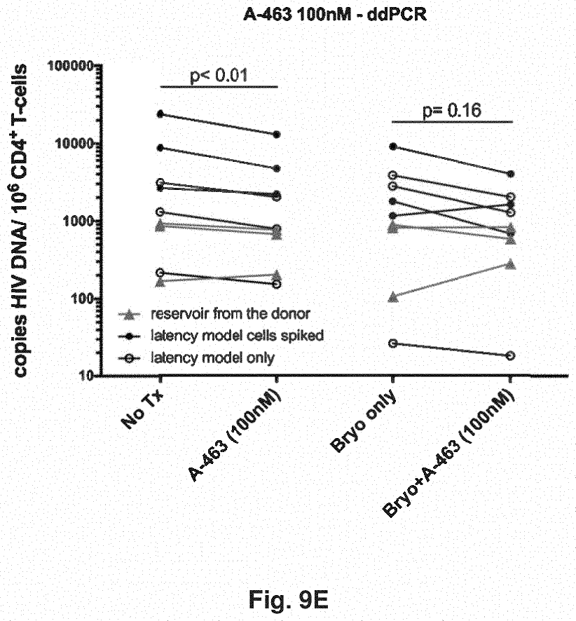

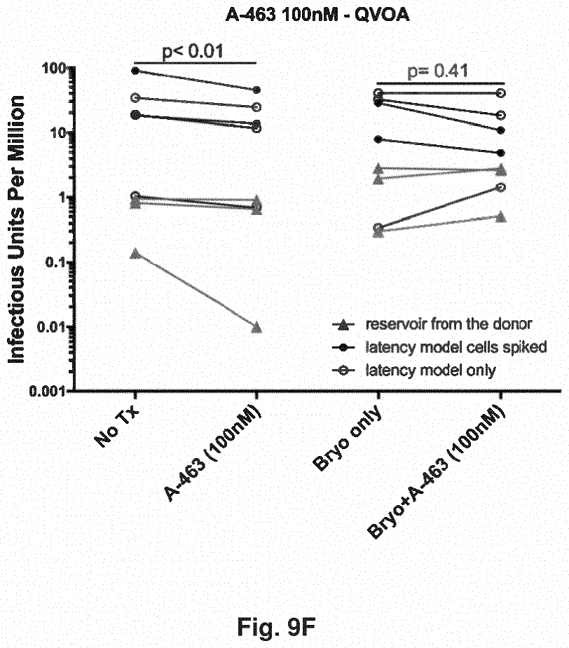

ABT-199 at 1 μM showed cell cytotoxicity, which resulted in not enough cells to measure replicative competent in ABT-199 (1 μM) only treatments (FIG. 9A). However, during treatments with a combination of Bryostatin (10 nM) and ABT-199 (1 μM), almost a significant decrease (p=0.06) on infectious units per million cells (IUPM) (FIG. 9B) was observed. A lower concentration of ABT-199 (100 nM) was also tested, which showed promising results: ABT-199 (100 nM) only showed significant reduction on IUPM (p=0.03) (FIG. 9D) and total HIV DNA copies (p=0.03) (FIG. 9C).

A-463 only treatments exhibited statistically significant reduction on both total HIV DNA copies (p<0.01) and infectious replicative competent (IUPM) (p<0.01), in all three models (FIG. 9E and FIG. 9F).

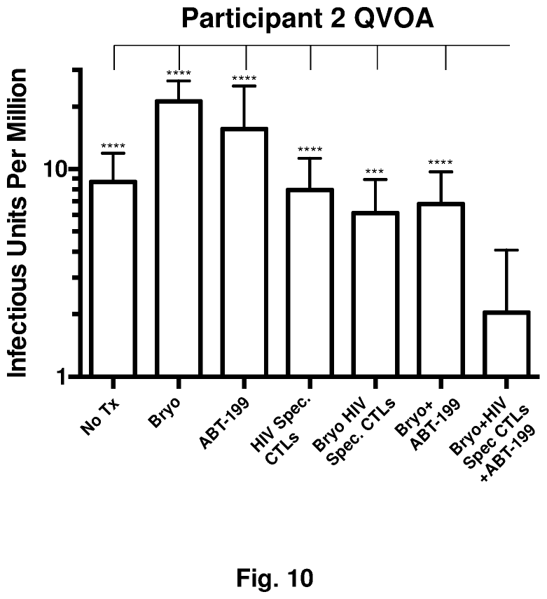

ABT-199 was then tested in a ‘spiked’ HIVE assay where HIV latency reservoir models were spiked into natural reservoirs (FIG. 10). This mixed population of cells was then treated with a combination of Bryostatin (10 nM), ABT-199 (100 nM), and HIV-specific CTLs. Treatment with either Bryostatin or ABT-199 alone, Bryostatin+ABT-199, or Bryostatin+HIV-specific CTLs did not lead to significant decreases in IUPM compared to the NoTx condition, whereas treatment with Bryostatin, ABT-199, and HIV-specific CTLs together led to significant decreases in IUPM compared to all other treatment conditions (**** p<0.0001, *** p<0.001)

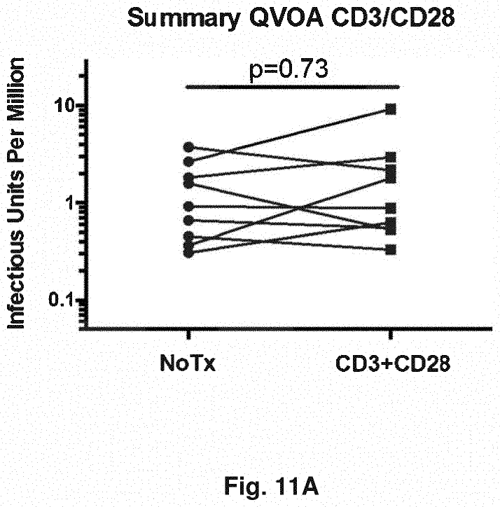

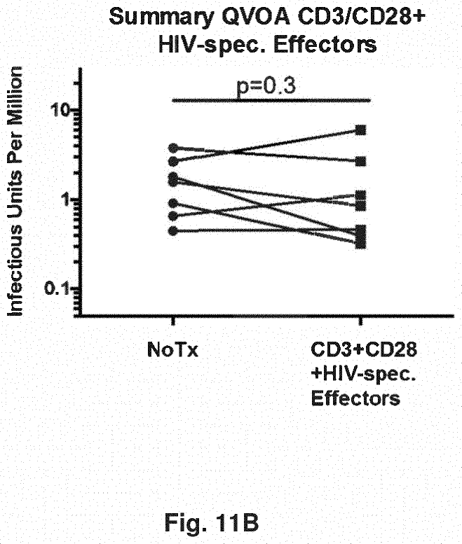

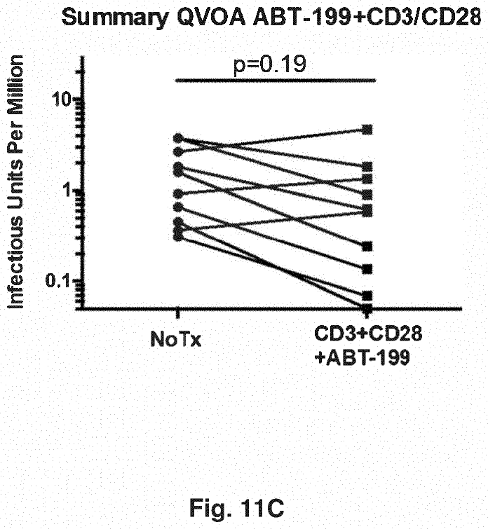

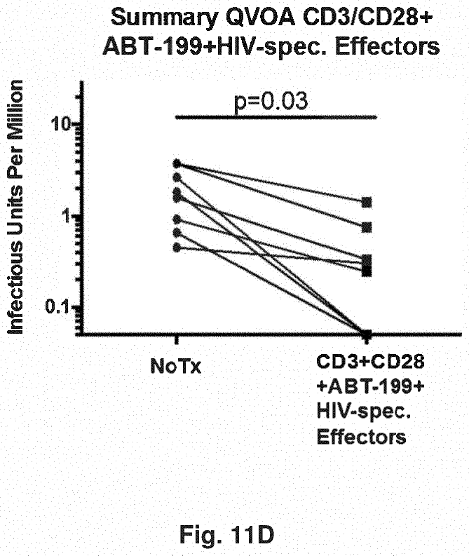

ABT-199 was then tested in a series of HIVE assays, where resting CD4⁺ T cells directly from HIV⁺ donors on long-term ART were treated with combinations of CD3/CD28 antibodies, ABT-199, and HIV-specific CTL effectors (FIG. 11). Treatment efficacy was assessed by comparison to a NoTx control. Treatment with CD3/CD28 antibodies did not lead to significant decreases in IUPM (p=0.73, FIG. 11A), and neither did treatment with CD3/CD28 antibodies in combination with HIV-specific CTLs (p=0.3, FIG. 11B). Treatment with CD3/CD28 antibodies in combation with ABT-199 also did not lead to consistent decreases in IUPM (p=0.19, FIG. 11C). Lastly, treatment with CD3/CD28 antibodies, ABT-199, and HIV-specific CTLs together led to consistent, significant decreases in IUPM (p=0.03, FIG. 11D). Critically, we find that decreases in IUPM are only seen when combining LRA with ABT-199 and a HIV-specific CTL effector, but not when combining only any two of these treatments.

It is to be understood that the foregoing embodiments and exemplifications are not intended to be limiting in any respect to the scope of the disclosure, and that the claims presented herein are intended to encompass all embodiments and exemplifications whether or not explicitly presented herein.

All patents and publications cited herein are fully incorporated by reference in their entirety.

What is claimed is:

1. A method of reducing or eliminating HIV reservoirs in a human infected with HIV, the method comprising administering to the human a therapeutically effective amount of a Bcl-2 family inhibitor and
(a) a therapeutically effective amount of a latency reversing agent;
(b) a therapeutically effective vaccine which enhances CD8+ T-cell responses;
(c) a therapeutically effective vaccine which enhances CTL responses;
(d) a therapeutically effective immunotherapy which enhances CD8+ T-cell responses; or
(e) a therapeutically effective immunotherapy which enhances CTL responses.

2. A method of reducing or eliminating HIV reservoirs in a human infected with HIV, the method comprising administering to the human a therapeutically effective amount of a Bcl-2 family inhibitor, a therapeutically effective amount of a latency reversing agent, and
(a) a therapeutically effective vaccine which enhances CD8+ T-cell responses;
(b) a therapeutically effective vaccine which enhances CTL responses;
(c) a therapeutically effective immunotherapy which enhances CD8+ T-cell responses; or
(d) a therapeutically effective immunotherapy which enhances CTL responses.

3. The method of claim 1 or 2, wherein the Bcl-2 family inhibitor is a Bcl-2 inhibitor.

4. The method of claim 4, wherein the Bcl-2 inhibitor is a selective Bcl-2 inhibitor.

5. The method of claim 1 or 2, wherein the Bcl-2 family inhibitor is a Bcl-xL inhibitor.

6. The method of claim 5, wherein the Bcl-xL inhibitor is a selective Bcl-xL inhibitor.

7. The method of claim 1 or 2, wherein the Bcl-2 family inhibitor is a selective Bcl-2/Bcl-xL inhibitor.

8. The method of claim 1 or 2, wherein the Bcl-2 family inhibitor is selected from the group consisting of ABT-263, ABT-737, sabutoclax, AT-101, TW-37, gambogic acid, BH31-1, ABT-199, obatoclax, HA14-1, A-1155463, A-1331852, WEHI-539, A-1210477, and UMI-77.

9. The method of claim 8, wherein the Bcl-2 family inhibitor is ABT-199.

10. The method of claim 8, wherein the Bcl-2 family inhibitor is A-1155463 or A-1331852.

11. The method of any one of claims 1-10, wherein the latency reversing agent is selected from the group consisting of protein kinase C activator, histone deacytelase inhibitor, Toll-like receptor 2 agonist, gamma chain cytokine, and P-TEFb inhibitor.

12. The method of any one of claims 1-10, wherein the latency reversing agent is selected from the group consisting of bryostatin 1, prostratin, ingenol B, vorinostat, romidepsin, panobinostat, Pam3CSK4, ALT-803, IL-2, IL-7, IL-15, heterodimeric IL-15, IL-15N72D-IL-15 receptor alpha Su/Fc fusion protein, JQ1, and I-BET151.

13. The method of any one of claims 1-11, wherein the latency reversing agent is a protein kinase C activator.

14. The method of claim 13, wherein the protein kinase C activator is bryostatin 1.

15. The method of any one of claims 1-14 further comprising administering a therapeutically effective amount of a cytotoxic T-lymphocyte to the human.

16. The method of any one of claims 1-14 further comprising administering a therapeutically effective amount of CD8+ T-cells to the human.


SEQUENCE LISTING (8 sequences)

SEQ ID NO: 1 - DNA, 18 bp, Artificial Sequence
  RPP30 - fprimer
gatttggacctgcgagcg

SEQ ID NO: 2 - DNA, 18 bp, Artificial Sequence
  RPP30-rprimer
gcggctgtctccacaagt

SEQ ID NO: 3 - DNA, 16 bp, Artificial Sequence
  RPP30-probe VIC
ctgaactgaaggctct

SEQ ID NO: 4 - protein, 6 aa, Artificial Sequence
  RPP30-probe VIC
MGBNFQ

SEQ ID NO: 5 - DNA, 18 bp, Artificial Sequence
  HIV-gag - fprimer
tactgacgctctcgcacc

SEQ ID NO: 6 - DNA, 17 bp, Artificial Sequence
  RPP30 -rprimer
tctcgacgcaggactcg

SEQ ID NO: 7 - DNA, 18 bp, Artificial Sequence
  probe FAM-
ctctctccttctagcctc

SEQ ID NO: 8 - protein, 6 aa, Artificial Sequence
  probe FAM-
MGBNFQ